(19) United States
(12) Patent Application Publication
(10) Pub. No.: US 2010/0239655 A1
(43) Pub. Date: Sep. 23, 2010

(54) TAURINE-BASED COMPOSITIONS AND THERAPEUTIC METHODS

(76) Inventor: Georgia Levis, New York, NY (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/749,844

(22) Filed: Mar. 30, 2010

Related U.S. Application Data
(63) Continuation-in-part of application No. 11/296,112, filed on Dec. 7, 2005, now abandoned.
Application No. 11/296,112 is a continuation-in-part of application No. 12/108,610, filed on Apr. 24, 2008.
(60) Provisional application No. 60/634,626, filed on Dec. 9, 2004.
(60) Provisional application No. 60/913,546, filed on Apr. 24, 2007.

(51) Int. Cl. A61K 9/127; A61K 31/185; A61K 31/59; A61K 31/513; A61K 31/196; A61P 17/00; A61K 31/56

(52) U.S. Cl. 424/450; 514/578; 514/167; 514/274; 514/567; 514/171

(57) ABSTRACT

Taurine and derivatives thereof may be used in conjunction with agents that activate or up-regulate the Taurine Transporter so as to enhance hydration of the skin and repair the skin bather, prevent apoptosis and oxidative damage in the skin, and heal or prevent photo-induced skin damage. The presence of biogenic taurine or taurine derivatives in tissues and/or body fluids can be used as a biomarker for tissue trauma. Detection and/or quantification of such biogenic taurine or derivatives form the basis for an assay for determining the presence and/or extent of tissue damage.

### CROSS-REFERENCE TO RELATED APPLICATIONS

This application is a continuation-in-part of U.S. patent application Ser. No. 11/296,112 filed Dec. 7, 2005 which claims priority of U.S. Provisional Patent Application Ser. No. 60/634,626 filed Dec. 9, 2004, entitled “Taurine-Based Compositions, Therapeutic Methods, and Assays”. This application is also a continuation-in-part of U.S. patent application Ser. No. 12/108,610 filed Apr. 24, 2008, entitled “Taurine-Based Methods and Compositions for the Treatment of Skin Conditions”, which claims priority of U.S. Provisional Patent Application Ser. No. 60/913,546 filed Apr. 24, 2007.

### FIELD OF THE INVENTION

This invention relates generally to the diagnosis and treatment of diseases. More specifically, the invention relates to compositions and methods for the treatment and prevention of dermatological diseases, based upon the use of taurine and its derivatives. More specifically, the invention relates to the use of topically applied taurine in combination with an agent which disrupts the skin barrier so as to activate the Taurine Transporter (TauT) system and promote hydration of the skin. The invention further relates to the use of taurine as a biomarker for tissue stress and damage.

### BACKGROUND OF THE INVENTION

Taurine is a biomolecule which contains a sulfonate group and an amine group. As such, taurine is broadly classified as an amino acid even though it does not include a carboxylate function. Taurine is not incorporated into protein, but it is present in high concentrations in mammalian plasma and cells and plays an important role in a number of essential biological processes such as the development of the central nervous system and the retina, calcium modulation, membrane stabilization, reproduction and immunity. Owing to relatively low levels of the enzyme cysteinesulfinic acid decarboxylase, the level of biosynthesis of taurine in humans is low. Taurine occurs naturally in animal-derived foods, particularly meats and seafood.

Taurine has previously been used in various therapies and utilitarian compositions. For example, nutritional supplements often include taurine. Taurine is known to function as a chelating agent for divalent ions such as calcium, and as such has been utilized for that purpose in various topical compositions. For example, U.S. Pat. No. 5,866,142 discloses the use of taurine as a chelating agent for calcium ions in an exfoliating composition. U.S. Pat. No. 5,869,068 similarly relies upon the ionic interactions of taurine to provide a composition in which the taurine acts as an agonist for chloride ion transport channels in cells, and thereby produces a slackening of the skin which reduces the appearance of wrinkles. U.S. Pat. No. 6,562,802 discloses compositions in which taurine is combined with other chelating molecules such as chitosan to promote wound healing. Taurine has also been reacted with silicone polymers to produce a material having a surfactant property, and U.S. Pat. Nos. 5,280,099 and 5,286,830 disclose such compositions and their various uses. While taurine has been noted to have some physiological effects, both internally and topically, its full range of utility has not been heretofore understood or exploited.

In accord with the present invention it has been found that taurine has a number of beneficial effects on the skin and nails, and these effects are of significance insofar as the skin is the largest organ of the body and serves a diverse group of functions. One of the major functions of the skin is to provide a barrier to harmful external factors such as environmental agents, infectious agents, and solar radiation. The skin also plays a key role in water homeostasis and functions as a barrier to prevent water loss from the body. This skin barrier function resides principally in the outer layers of the skin and is often thought of in terms of a brick wall in which the corneocytes and outer layer keratinocytes represent the bricks, the intercellular lipid matrices represent the mortar, the intracellular adhesion molecules represent reinforcing bars, and the hydrolipid film on the surface represents a sealant. Each of these functional constituents is important for optimal barrier function. Excessive water loss from the skin caused by dry (xerotic) and/or inflammatory skin conditions can lead to shrinkage of the corneocytes which will degrade the integrity of the skin barrier. As detailed herein, taurine-based materials can act to enhance the hydration of the skin and thereby repair the skin barrier.

In accord with the present invention, it has been found that taurine is taken up from the extracellular milieu by a high affinity, low capacity sodium and chloride coupled transport system referred to as the Taurine Transporter (TauT). The system is activated in response to hyperosmolar conditions in the skin, particularly in the stratum granulosum. The TauT acts to increase the intracellular concentration of taurine and thereby directly protect the cells against degradation and other stresses. It has been found that under normal physiological conditions, taurine is present in high concentrations in the keratinocytes of the granular and upper spinous layers of the skin, but not in the basal layer, lower spinous layer, or stratum corneum. There is a circulating pool of taurine in the epidermis released by keratinocytes before cornification. This taurine is subsequently reclaimed by nearby cells. In accord with the present invention, the TauT has been localized in the skin and found to be absent in the dermis as well as in the basal layer or stratum corneum, but present in high levels in the outermost granular layer, and to a lesser degree in the spinous layer. It has been found that the greatest degree of taurine activity is in the stratum granulosum, and this finding is consistent with the anatomic position of the stratum granulosum as the outermost layer of living keratinocytes, which cells are the most vulnerable to environmental fluctuations in humidity. The distribution of taurine and the TauT has also been found to be consistent with the presence of a high water gradient across the stratum granulosum from 30% water content at the stratum corneum-stratum granulosum interface to 70% within the stratum spinosum. This gradient exposes cells of the granular layer to a persistent hyperosmotic/xerotic stress. While taurine has been found to play a significant role in promoting the hydration of tissue, topical applications of taurine to the skin have produced less than expected degrees of hydration even when the taurine was employed in combination with propylene glycol, polysorbates, and other such nonionic surfactants, which are generally recognized in the art as being permeation enhancers. While such compositions do penetrate portions of the outer layers of the skin, it has been found in accord with the present invention that such compositions cannot provide for the delivery of taurine and taurine-related materials to deeper portions of the epidermis wherein the TauT system is active.

As will be described in detail hereinbelow, the present invention utilizes taurine or taurine-related materials in combination with one or more agents which disrupt the skin barrier so as to activate the TauT system and promote the stable hydration of the skin tissues. Use of a skin barrier disrupting agent is counterintuitive in therapies having as their object the restoration and maintenance of the skin barrier. However, the inventor hereof has demonstrated that such disruption is critical to the activation of the TauT system and, provided there is an available exogenous source of taurine, thereby enhances skin hydration and overall skin barrier quality.

In accord with a still further aspect of the present invention, it has been found that taurine is a very effective biomarker for tissue trauma; hence, measurement of taurine levels in tissues or body fluids can be used to determine existence and amount of tissue trauma an organism has experienced. These and other aspects of the present invention will be described in detail hereinbelow.

### SUMMARY OF THE PRESENT INVENTION

Disclosed herein is a method for enhancing the hydration of the skin. The method comprises applying to the skin a composition which includes an agent which activates the TauT system (by temporarily disrupting the skin barrier) in combination with an exogenous source of taurine or a derivative thereof. This combination allows the activating material to up-regulate TauT raising intracellular taurine concentrations and promoting and maintaining the hydration of the skin. The skin barrier disrupting agent may be a chemical agent such as an ionic surfactant, or it may be a physical agent such as a microinjector array, an ultrasonic transducer, an electronic field generator, a pressure transducer, or the like.

In particular embodiments, the derivative of taurine is selected from the group consisting of salts, esters, complexes and conjugates of taurine. In a specific instance, the derivative of taurine comprises a chloramine/taurine complex. The composition may further include active ingredients such as retinoids, emollients, topical anesthetics, moisturizers, corticosteroids, permeation enhancers, sunscreens, antibiotics, coloring agents, fragrances and combinations thereof. In other instances, the composition may also include vitamin D, analogues of vitamin D, calcineurin inhibitors, an immunomodulating drug, an anticancer agent, 5-FU, diclofenac and combinations thereof. The compositions may also include a vehicle comprising materials such as physiological lipids, non-physiological lipids, phospholipids, triglycerides, diglycerides, monoglycerides, free fatty acids, fatty alcohols, ceramides, cholesterol, cholesterol esters, isoflavonoids and various combinations thereof. In specific instances, the concentration of taurine in the composition is in the range of 1-20 weight percent.

Also disclosed is a method for repairing the skin barrier. The method comprises applying to the skin a topical composition which comprises taurine or a derivative thereof. Further ingredients as discussed above may also be included in the composition.

In yet another embodiment of the present invention, there is disclosed a method for preventing photo damage to the skin by applying a composition which inhibits apoptosis in the skin. The active agent for inhibiting apoptosis may comprise taurine or a derivative thereof. The composition may further include an ultraviolet light absorbing or reflecting sunscreen material.

Also disclosed are methods for inhibiting aging of the skin by applying a composition which comprises taurine or a derivative thereof.

Further disclosed is a method for treating diseases associated with the nails by applying a composition which includes taurine or a derivative thereof to the nails. Further disclosed is a method for detecting and/or quantifying trauma to the tissue of a patient's body. The method comprises the step of assaying a tissue or body fluid of the patient to determine the presence and/or quantity of taurine or its derivatives in the tissue or fluid. Further disclosed are specific compositions for practicing the methods of the present invention.

### DETAILED DESCRIPTION OF THE INVENTION

In accord with the first aspect of this invention, it has been found that the topical application of taurine and its derivatives in combination with an agent which disrupts the skin barrier is of significant benefit in treating a number of dermatological conditions. It is significant that taurine is a material which carries the designation “generally regarded as safe.” Within the context of this patent “taurine” is understood to also include derivatives of taurine such as its salts and esters as well as its complexes, conjugates and the like; and specifically includes biogenic taurine derivatives such as chloramine/taurine complexes, as well as synthetic and semi-synthetic derivatives. Within the context of this patent, all references to “taurine” are understood to also include derivatives of taurine.

One embodiment of the taurine-based therapy of the present invention involves repair of the skin barrier. The epidermis forms a barrier between the moisture-rich tissues of an individual's body and the relatively dry exterior environment. The outermost layer of the skin, the stratum corneum which is largely composed of corneocytes derived from epidermal keratinocytes, is constantly exposed to water loss placing it under persistent osmotic stress. Taurine is found in the epidermis of humans and a number of other animal species, and it is believed that the dermal cells counter water loss by increasing the amount of taurine and other such osmolytes therein. It has also been found that biogenic taurine is actively transported into the epidermis where it is believed to function to protect cells from dehydration and other stressors, and it has been determined that taurine accumulation in the skin is increased under induced osmotic stress.

In accord with the present invention, it has been found that taurine exerts its beneficial effect with regard to the skin by activation of the Taurine Transport (TauT) system. Further in accord with the present invention, it has been found that for taurine to exert its maximum effect, it must be bioavailable to the TauT system. As detailed above, the TauT has been found to be present in high levels in the outermost granular layer and to a lesser degree in the spinous layer of the skin with the greatest degree of taurine activity to be found in the stratum granulosum. The TauT system has been found to be absent in the dermis and basal layer of the stratum corneum. As further discussed above, the outer layers of the skin, namely corneocytes and outer layer keratinocytes, provide a very effective barrier shielding inner layers of the skin from external stressors. These layers do not include significant expression of TauT and furthermore provide a barrier preventing externally applied materials such as taurine materials from penetrating and interacting with the TauT system which lies in the deeper skin layers.

Typical dermatological compositions have been formulated to penetrate and soften the outer layers of the skin, and in that regard they include penetration enhancers such as propylene glycol, polysorbates, and similar such materials. However, such compositions are specifically formulated not to significantly disrupt the skin barrier, it being commonly accepted that such disruption of the skin barrier is detrimental to the objective of maintaining hydration or otherwise conditioning the skin. Hence, prior art compositions that do not incorporate materials that induce or up-regulate the expression of the TauT system are ineffective in making exogenously applied taurine bioavailable to the osmotically stressed cells of the outer epidermis and are ineffective in promoting access of exogenous taurine to the TauT system.

In contrast to the prior art, the present invention promotes the hydration of the skin and fosters the repair and maintenance of the skin barrier by the use of a skin barrier disrupting agent which activates the Taurine Transporter system, in combination with a taurine material. The disrupting agent allows the taurine material to reach the TauT system, to be sequestered by the system and transported to the osmotically stressed cells of the outer skin thereby promoting and maintaining hydration of the skin cells and restoring skin barrier function. Use of an agent that temporarily disrupts the skin barrier is counterintuitive insofar as the prior art believes that such agents would be detrimental to the maintenance and repair of the skin barrier.

In accord with the present invention, agents for facilitating the biological function of the TauT system will typically comprise taurine or other taurine-related species as described herein.

The agent for temporarily disrupting the skin barrier will, in specific instances, comprise a chemical agent. One group of such agents comprises ionic surfactants. As discussed above, the prior art disfavors the use of ionic surfactants in compositions for the treatment of skin conditions since such surfactants are known to disrupt skin barrier function and produce drying and other such damage. In other instances, controlled physical disruption of the skin barrier may be utilized in the practice of the present invention. Such disruption may be achieved through the use of micro needle arrays of the type shown, for example, in U.S. Pat. No. 6,256,533, the disclosure of which is incorporated herein by reference. Likewise, disruption may be accomplished by application of ultrasonic energy or an electrical field to the skin. In yet other instances, disruption may be accomplished mechanically by the use of pressure. Such disruption both activates or up-regulates the TauT system and allows penetration of the co-administered Taurine material to the tissue compartments where the TauT system resides. In dry skin conditions, this activation or so-called up-regulation of the TauT system coupled to the bioavailable administration of exogenous taurine (through the otherwise impervious outer layers of the skin) provides the necessary prerequisites for active transport of taurine into the taurine depleted, osmotically stressed cells of the outer epidermis. Increased intracellular taurine leads to re-hydration in these tissues and to restoration of skin barrier function.

In those instances where the disrupting agent is a chemical agent, it may be compounded directly into a single formulation with taurine material which is applied to the skin. In other instances, the chemical disruption agent may be separately applied to the skin either before or after the taurine material. In those instances where physical disruption is employed, disruption of the skin barrier may take place either before, during, or after the application of the taurine material to the skin.

In view of the foregoing, the present invention provides for enhanced skin barrier protection by the use of externally applied taurine supplements in conjunction with skin barrier disrupting agents that cause the up-regulation of the Taurine Transporter system. As such, taurine functions as a humectant which increases the hydration of dehydrated, or potentially dehydrated, skin so as to enhance and maintain the integrity of the skin barrier. In accord with the present invention, there are provided topical compositions of taurine in a carrier vehicle that induces Taurine Transporter activation or up-regulation, and these compositions function to rehydrate the skin, prevent moisture loss and restore the integrity of the skin barrier.

The concentration of taurine used in various activating preparations of the present invention will vary depending upon the specific application, the type of carrier vehicle, and other such factors. However, in one group of embodiments, the concentration of taurine in the topical preparation is in the general range of 1-20 weight percent. One particular group of compositions has a concentration in the range of 5-15 percent; another group of compositions includes approximately 1-5 percent by weight of taurine. In specific compositions, concentrations of approximately 2.5 percent by weight are employed.

The carrier vehicle for the various compositions of the present invention may comprise an aqueous, lotion, gel, or a lipid-rich emollient vehicle as is well known in the art. In one group of embodiments, the carrier is a liposomal, or other lamellar or multi-lamellar, vehicle. It is to be understood that ancillary ingredients may be included in the compositions of the present invention. These ancillary ingredients can include dermally active drugs such as vitamin D and its analogues, calcineurin inhibitors and other immunomodulating drugs, anticancer agents and other drugs indicated for actinic keratosis, including, but not limited to, 5-FU, diclofenac and the like, as well as other substances known to repair and maintain the skin barrier function. Such substances include, but are not limited to: physiological lipids, such as essential and non-essential fatty acids, phospholipids, ceramides, cholesterol and its esters, isoflavonoids and other protease inhibitors. In the instance where skin barrier repair is contemplated, these ancillary ingredients can also include active ingredients such as retinoids, corticosteroids, antibiotics and the like. The ancillary ingredients may also include emollients, topical anesthetics, permeation enhancers, sunscreens, colors, fragrances and other such materials as is known in the art.

In accord with another aspect of the present invention, it has been found that taurine can protect normal human epidermal keratinocytes from ultraviolet induced cell death (apoptosis), even at micromolar concentrations. This effect is independent from and in addition to, the skin barrier repair and hydrating effects taurine exerts on dermal cells.

In accord with this particular aspect of the present invention, taurine-based compositions of the type described hereinabove are effective for both preventing and healing photo-induced skin damage. Given the fact that taurine itself is not a very strong absorber of those ultraviolet wavelengths which contribute to skin tanning, compositions of taurine may be used to permit tanning while preventing various types of skin damage. In other instances, taurine compositions may include ultraviolet absorbing or reflecting sun block agents of the type known in the art. Also, as described above, the compositions may include further therapeutic and non-therapeutic materials.

Taurine's actions in repairing the skin barrier, restoring hydration to the skin, preventing apoptosis, and preventing oxidative damage to tissue will cause it to have utility in compositions for preventing or alleviating chronological or photo aging of the skin. In this regard, taurine may be incorporated into cosmetics as well as therapeutic preparations such as lotions and the like.

It is to be understood that within the context of this disclosure, skin is understood to include mucosal tissues. The compositions of the present invention will have utility for hydrating and restoring mucosal tissues. For example, the taurine-based compositions may be used on buccal or vaginal tissues, and will also have utility in the ano-rectal region for treating inflammations, hemorrhoids, fissures, and other conditions.

Because of the presence of the sulfonate moiety, taurine and its derivatives can interact with the keratinous matrix of the nails so as to enhance their permeability and facilitate the transport of materials into and through the nails. Furthermore, taurine and various of its derivatives have a direct antimicrobial effect. Hence, taurine, used either alone or with other therapeutic materials such as antifungal drugs, can be effective in treating onychomycosis, psoriatic nail disorders, and other diseases associated with the nails.

While the foregoing has primarily described compositions based upon the free amino acid taurine, it is to be understood that derivates of taurine may likewise be employed in the practice of the present invention. For example, taurine may be utilized in the form of a salt or an ester. Taurine may also be employed in the form of various conjugates, and given the chemical reactivity of the sulfonate and amine group, a number of such conjugates will be readily apparent to those of skill in the art. For example, taurolidine is a taurine-based conjugate based upon taurine and formaldehyde. This material is known in the art, and has been used as an antibacterial material, and may be utilized in the practice of various aspects of the present invention either with or without free taurine. Chloramine/taurine complexes may be similarly employed.

In view of the teaching presented herein, one of skill in the art could readily formulate various topical compositions of taurine. One specific composition comprises, on a weight basis:

| | |
|---|---|
| Taurine | 2.5% |
| Cetostearyl alcohol | 7.2% |
| Ceteth-20 | 1.8% |
| Light mineral oil | 6% |
| White petrolatum | 15% |
| Propyl paraben | 0.15% |
| Trolamine (99%) | 1% |
| Triethanolamine lauryl sulfate | 0.25% |
| Purified water | 66.6% |

The foregoing is a cream-based taurine composition. It may be used as a skin barrier repair product, moisturizer or skin protectant and can function to alleviate photo-induced skin damage, inhibit oxidative skin damage, and inhibit apoptosis. Further ingredients as noted above, as well as ancillary ingredients such as fragrances, coloring agents and the like, may be incorporated thereinto. Likewise, the composition may be formulated as a lotion, an emollient or fatty cream or ointment, gel, foam or other pharmaceutically acceptable vehicle for topical use. For example, preparations used to prevent sun damage will preferably be made as low viscosity lotions or as water-based compositions.

A composition similar to the foregoing was prepared utilizing 1.5 weight percent taurine. Both of the compositions were evaluated in a patient having both psoriasis and eczema. Both creams were found to be excellent moisturizers for xerotic skin. Neither was irritating to dry or normal skin, nor did either irritate the psoriasis or eczema. Both the psoriasis and eczema improved within the 1.5% and 2.5% creams, thereby demonstrating that in addition to the skin barrier repair and moisturizing effect, taurine has anti-inflammatory qualities.

In accord with yet another aspect of the present invention, it has been found that taurine levels in various tissues increase as a result of physical damage or other trauma; and biogenic taurine can be a marker for such trauma. Specifically, the detection of elevated levels of taurine or its derivatives will suggest that an organism has suffered some tissue trauma. Likewise, quantification of the level of taurine or its derivatives can be used to estimate the severity of the trauma. Taurine is very water soluble, and relatively simple assays based upon the analysis of body fluids such as saliva, urine, sweat or blood for taurine can be employed. Also, levels of taurine measured in tissue samples can provide very specific information about the physiological state of that tissue. High performance liquid chromatography (HPLC) is particularly effective for detecting and quantifying levels of taurine and its derivatives in a variety of fluids. Taurine levels may be measured by a number of other analytical techniques well known in the art including immunochemical techniques as well as more classical types of chemical reagent interactions.

The present invention provides a number of therapeutic compositions and methods based upon taurine-containing topical preparations. Also, the present invention provides for an analytical method for detecting and/or quantifying tissue trauma based upon the measurement of taurine levels. Some specific descriptions and examples have been presented herein. In view of this teaching, yet other embodiments and versions of the present invention will be readily apparent to those of skill in the art. The foregoing discussion and description is illustrative of specific embodiments of the invention, but is not meant to be a limitation upon the practice thereof. It is the following claims, including all equivalents, which define the scope of the invention.

1. A method for the treatment of the skin, said method comprising:
applying to the skin a composition comprising a material selected from the group consisting of taurine and/or a derivative of taurine, said material being biologically suitable and available for uptake by the Taurine Transporter (TauT) system; and
contacting the skin with a chemical agent which disrupts the skin barrier to cause activation or up-regulation of the TauT system; whereby said taurine material is enabled to cross the skin bather and be taken up by the TauT system.

2. The method of claim 1, wherein said agent which disrupts the skin barrier is a chemical agent.

3. The method of claim 2, wherein said chemical agent is an ionic surfactant.

4. The method of claim 2, wherein said chemical agent is azone.

5. The method of claim 1, wherein said agent which disrupts the skin barrier is applied to the skin at the same time as is the taurine material.

6. The method of claim 1, wherein said agent which disrupts the skin barrier is applied to the skin prior to the time the taurine material is applied thereto.

7. The method of claim 1, wherein said agent which disrupts the skin barrier is applied to the skin after the time the taurine material is applied thereto.

8. The method of claim 1, wherein said derivative of taurine is selected from the group consisting of salts, esters, complexes and conjugates of taurine.

9. The method of claim 1, wherein said composition further includes a member selected from the group consisting of: retinoids, emollients, topical anesthetics, moisturizers, corticosteroids, sunscreens, antibiotics, coloring agents, fragrances and combinations thereof.

10. The method of claim 1, wherein said composition further includes a member selected from the group consisting of: vitamin D, analogues of vitamin D, calcineurin inhibitors, an immunomodulating drug, an anticancer agent, diclofenac and combinations thereof.

11. The method of claim 1, wherein said composition further includes a member selected from the group consisting of physiological lipids, non-physiological lipids, phospholipids, ceramides, cholesterol, cholesterol esters, isoflavonoids, protease inhibitors, and combinations thereof.

12. The method of claim 1, wherein said vehicle includes liposomes.

13. The method of claim 1, wherein the concentration of taurine or said taurine derivative in said composition is in the range of 1-20 weight percent.

14. A method for detecting and/or quantifying trauma to tissue of a patient's body, said method comprising the step of:
assaying a tissue or body fluid of said patient to determine the presence and/or quantity of taurine or its derivatives in said tissue or fluid.

15. The method of claim 14, wherein said body fluid is selected from the group consisting of blood, urine, saliva, sweat, or combinations thereof.

16. The method of claim 14, wherein said tissue comprises skin, lungs, gastrointestinal tissue, and muscle.
